(19) United States
(12) Patent Application Publication
(10) Pub. No.: US 2020/0152332 A1
(43) Pub. Date: May 14, 2020

(54) SYSTEMS AND METHODS FOR DYNAMIC MONITORING OF PATIENT CONDITIONS AND PREDICTION OF ADVERSE EVENTS

(71) Applicant: KONINKLIJKE PHILIPS N.V., EINDHOVEN (NL)

(72) Inventors: YANG YANG, MEDFORD, MA (US); TIANZHONG YANG, EINDHOVEN (NL); REZA SHARIFI SEDEH, MALDEN, MA (US); Yugang Jia, Wichester, MA (US)

(21) Appl. No.: 16/619,293

(22) PCT Filed: Jun. 5, 2018
(86) PCT No.: PCT/EP2018/064685

§ 371 Date: Dec. 4, 2019

Related U.S. Application Data
(60) Provisional application No. 62/518,033, filed on Jun. 12, 2017.

(51) Int. Cl. G16H 50/30; G06F 16/35; G16H 50/20

(52) U.S. Cl. CPC G16H 50/30; G16H 50/20; G06F 16/35

(57) ABSTRACT

Systems and methods are provided for healthcare predictive analysis based on dynamic monitoring of patient conditions. Dynamic monitoring is used by healthcare provider entities to collect historical claim feed data regarding its patients. The historical claim feed data is used to monitor patients' progress and conditions. Moreover, this data is used to train and update a predictive model used to predict the occurrence of events. The model predicts the occurrence of events using a sliding window-based algorithm, in which subsets (e.g., windows) of the historical claim feed data are sequentially used to train the model. For each window of data, the model extracts features and outcomes, and trains the model based thereon. The model then extracts features and outcomes of the next window of data and updates the existing model based thereon. The resulting model is run against a set of a data to predict the occurrence of events.

### FIELD

The present application generally relates to providing healthcare analytics, and more specifically to systems and methods for dynamically monitoring healthcare and predicting the occurrence of events.

### BACKGROUND

Healthcare provider entities are hospitals, institutions and/or individual practitioners that provide healthcare services to individuals. In recent years, there has been an increased focus on monitoring and improving the delivery of healthcare around the globe, and doing so in the most cost effective manner possible. Traditionally, healthcare delivery has been driven by volume, meaning that healthcare delivery entities are motivated to increase or maximize the volume of healthcare services, visits, hospitalizations and tests that they provide.

More recently, there is a growing trend in which healthcare delivery is shifting from being volume driven to being outcome or value driven. This means that healthcare provider entities are being incentivized to provide high quality healthcare while minimizing costs, rather than simply providing the maximum volume of healthcare. One way in which healthcare delivery entities are being incentivized is by the implementation of payment systems (e.g., Accountable Care Organizations (ACOs)), in which groups of healthcare provider entities cooperate to provide coordinated high quality care, and are paid according to a pay-for-performance model.

This shift to outcome or value driven service has thus increased the importance of monitoring and measuring healthcare data to achieve safe, effective, patient-centered, timely, efficient and equitable healthcare delivery. Effective monitoring and measuring of healthcare data provides patient oversight and the ability to predict the probability or likelihood of the occurrence of healthcare related events, such as adverse events.

Thus, monitoring healthcare data and predicting events is becoming an increasingly important component in the business of healthcare delivery by healthcare provider entities. Members, staff, directors and officers (e.g., chief financial officers (CFOs), chief executive officers (CEOs)) of healthcare provider entities are thus tasked with dynamically and effectively monitoring healthcare data and accurately predicting the occurrence of healthcare related events.

However, current healthcare monitoring and predictive analysis is limited by, among other things, the shortcomings of existing healthcare datasets including their lack of particularity and their staleness, the complexity and high cost of obtaining the data, and the rigidity of existing models. For instance, existing healthcare domain datasets each have limitations that prevent or hamper the ability to efficiently and cost-effectively compile an optimal dataset that can be used to provide precise predictive analyses. The Healthcare Cost and Utilization Project (HCUP) is a set of healthcare databases developed through United States federal and state partnerships sponsored by the Agency for Healthcare Research and Quality (AHRQ). HCUP databases are however limited to in-patient, ambulatory and emergency department data only at a community granularity level rather than on the level of particular healthcare providers or groups of providers associated with an ACO. Moreover, the HCUPs data can be purchased and obtained for a given calendar year only after six to eighteen months after the end of that calendar year. Philips' eICU program collects and stores information related only to intensive care unit stays only. Electronic health record (EHR) databases contain health-condition-related information, but not detailed information relating to patients' visits to healthcare provider entities. Moreover, EHR datasets are typically not available in hospitals and similar entities, or the complexity of the hospitals' underlying information technology infrastructure prevents easy access to that data. These types of problems related to the type of data, and the cost and complexity of obtaining the data that is currently available, are common throughout existing healthcare databases.

In addition to the above-described shortcomings of existing healthcare datasets, current predictive models are inflexible and lack the currency needed to provide optimal predictive analyses. For example, the models employed by The Johns Hopkins Adjusted Clinical Groups (ACG) System and the Mayo Clinic Health System provide nationwide or global analytics. It is therefore not feasible or far too costly and complex to train these models to be particularized to provide predictive analysis for a specific hospital or other healthcare provider entity. Moreover, not only is the datasets used by these models not sufficiently localized, but due to their size, they are often not sufficiently up-to-date as is desirable to provide optimal predictions. Implementing and maintaining these types of global or nationwide models requires a large amount of coordination that further increases their complexity and cost.

There is a need therefore for improved systems and methods that dynamically monitor healthcare data such as patient health conditions, and predicts the occurrence of adverse events. There is a need for the data and conditions that are dynamically monitored to include timely and sufficiently specific details. There is also a need for data and conditions that are dynamically monitored to relate to particular healthcare delivery entities such that the occurrence of adverse events for, at or related to that healthcare delivery entity can be more accurately and precisely predicted.

### SUMMARY

The present application provides systems and methods for dynamic monitoring of patient conditions and prediction of adverse events.

In some embodiments, a healthcare predictive analysis system includes at least one memory and at least one processor. The at least one memory stores a set of historical data corresponding to a period of time previous to a present time at a time of execution. The at least one processor communicatively coupled to the at least one memory. A set of historical data is retrieved from the at least one memory. A plurality of windows is identified among the set of historical data, each of the plurality of windows being a subset of the set of historical data corresponding to a sub-period of time among the period of time. A current window is identified from among the plurality of windows. For each of the windows among the plurality of windows: a current set of features and outcomes corresponding to the current window is extracted, the current features being extracted from the sub-period of time corresponding to the current window and the current outcomes being extracted from a current outcomes sub-period of time subsequent to the sub-period of time corresponding to the current window; a current generation predictive model is trained based on the extracted current set of features and outcomes, the current generation predictive model corresponding to the current window; a next window is identified from among the plurality of windows, the next window being the next-in-time window relative to the current window; a next set of features and outcomes corresponding to the next window is extracted, the next features being extracted from the sub-period of time corresponding to the next window and the next outcomes being extracted from a next outcomes sub-period of time subsequent to the sub-period of time corresponding to the next window; a next generation predictive model is trained based on the current generation predictive model and the extracted next set of features and outcomes, the next generation predictive model corresponding to the next window; and the current window is substituted with the next window. A probability of an occurrence of one or more events is predicted using a predictive model corresponding to the current window on a subset of data corresponding to a predictive sub-period of time among the period of time.

In some embodiments, the set of historical data is claim feed data corresponding to a healthcare provider entity.

In some embodiments, at least a portion of the set of historical data is received from a third-party database.

In some embodiments, the portion of the set of historical data received from the third-party database is unstructured data, and the at least one processor is operable to structure the unstructured data.

In some embodiments, the sub-periods of time corresponding to the plurality of windows are the same length.

In some embodiments, the predictive model used to predict the probability of the occurrence of the one or more events corresponds to a window corresponding to the sub-period of time closest to the present time.

In some embodiments, each of the extracted current outcomes and next outcomes is associated with a time-to-event variable indicating a length of time from the start of the sub-period of time corresponding to the current window and the next window, respectively.

In some embodiments, the training of the current generation predictive model includes: for each of the extracted current outcomes: identifying, among the extracted current features, patterns related to the given extracted current outcome; identifying one or more current predictive variables based on the identified patterns related to the given extracted current outcome, each of the one or more current predictive variables being one of the extracted current features; and assigning weights to each of the one or more current predictive variables based on the identified patterns related to the given extracted current outcome. The training of the next generation predictive model includes: for each of the extracted next outcomes: identifying, among the extracted next features, patterns related to the given extracted next outcome; identifying one or more next predictive variables based on the identified patterns related to the given extracted next outcome, each of the one or more next predictive variables being one of the extracted next features; and assigning weights to each of the one or more next predictive variables based on the identified patterns related to the given extracted next outcome. If the given extracted next outcome matches one of the extracted current outcomes, the assigning of weights includes updating the weights of each of the one or more current predictive variables corresponding to the one of the extracted current outcomes that match the one or more next predictive variables corresponding to the one of the extracted next outcomes.

In some embodiments, predicting the probability of an occurrence of one or more events using the predictive model includes: for each of the one or more events: identifying one or more relevant outcomes in the predictive model; identifying the predictive variables related to each of the one or more relevant outcomes; identifying matching features in the subset of data corresponding to the predictive sub-period of time that match features corresponding to the identified predictive variables related to each of the one or more relevant outcomes; and calculating a probability of the occurrence of each of the one or more events based on the weights of the respective matching features.

In some embodiments, the predicting of the probability of the occurrence of one or more events is performed for a specified future date or date range.

In some embodiments, a testing error rate is calculated by executing the current generation model against the extracted next features and outcomes.

In some embodiments, a method is provided for healthcare predictive analysis, comprising: retrieving a set of historical data stored in at least one memory, the set of historical data corresponding to a period of time previous to a present time at a time of execution; identifying a plurality of windows among the set of historical data, each of the plurality of windows being a subset of the set of historical data corresponding to a sub-period of time among the period of time; identifying a current window from among the plurality of windows; for each of the windows among the plurality of windows: extracting a current set of features and outcomes corresponding to the current window, the current features being extracted from the sub-period of time corresponding to the current window and the current outcomes being extracted from a current outcomes sub-period of time subsequent to the sub-period of time corresponding to the current window; training a current generation predictive model based on the extracted current set of features and outcomes, the current generation predictive model corresponding to the current window; identifying a next window from among the plurality of windows, the next window being the next-in-time window relative to the current window; extracting a next set of features and outcomes corresponding to the next window, the next features being extracted from the sub-period of time corresponding to the next window and the next outcomes being extracted from a next outcomes sub-period of time subsequent to the sub-period of time corresponding to the next window; training a next generation predictive model based on the current generation predictive model and the extracted next set of features and outcomes, the next generation predictive model corresponding to the next window; and substituting the current window with the next window; and predicting a probability of an occurrence of one or more events using a predictive model corresponding to the current window on a subset of data corresponding to a predictive sub-period of time among the period of time.

### BRIEF DESCRIPTION OF THE DRAWINGS

The present application will be more fully understood from the following detailed description taken in conjunction with the accompanying drawings, in which:

FIG. 1 illustrates an exemplary embodiment of a healthcare environment including a healthcare analytics predictive system;

FIG. 2 is a flow chart illustrating an exemplary embodiment of a process for dynamically monitoring patient conditions and predicting events using the healthcare analytics predictive system of FIG. 1;

FIG. 3 illustrates an exemplary embodiment of a data model for storing healthcare data used by the predictive system of FIG. 1;

FIG. 4 illustrates an exemplary embodiment of a process for extracting features and outcomes from stored healthcare data;

FIG. 5A illustrates the extraction of features and outcomes in relation with a window of the healthcare data graphically illustrated as temporal data;

FIG. 5B illustrates the extraction of features and outcomes, and the prediction of events, in relation with another window of healthcare data graphically illustrated as temporal data; and

FIG. 6 illustrates a graphical representation of an exemplary embodiment of a process for dynamically monitoring patient conditions and predicting events.

### DETAILED DESCRIPTION

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of various embodiments generally have similar features when those components are of a similar nature and/or serve a similar purpose.

The example embodiments presented herein are directed to systems and methods for dynamically monitoring patient conditions and predicting adverse events. More specifically, the systems and methods provided herein describe the collection and storage of data by healthcare provider entities. Examples of such data include historical claim feed data, which is information relating to patients' medical claims. The data is used to dynamically monitor patient conditions by predicting the occurrence of events, including adverse events. To predict the occurrence of events, a model is trained using the historical claim feed data. The training of the model is performed using a sliding-window approach or algorithm, in which one window or a set of windows from the historical claim feed data are sequentially analyzed. That is, features and outcomes are extracted from the existing defined windows and a model is trained based on these. The existing model is updated using the extracted features and outcomes of the next coming window. Each window of data is sequentially used to update the model. The most up-to-date model is used to predict the occurrence of events at a future time.

### System

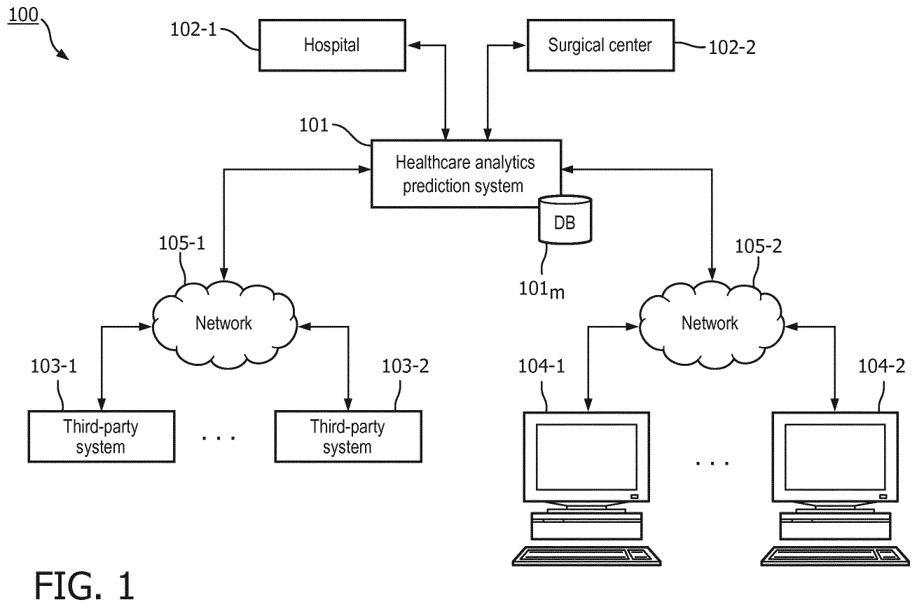

FIG. 1 illustrates a healthcare analytics environment 100, according to an exemplary embodiment. The healthcare analytics environment 100 includes a healthcare analytics prediction system 101 that is used to monitor patient health data and conditions, and predict the occurrence of adverse events. The healthcare analytics prediction system 101 includes one more memories and/or databases, such as database 101m. The database 101m can store healthcare related information for monitoring patients' health and conditions. The stored healthcare related information can also be used to predict adverse events. Although not illustrated, the healthcare analytics prediction system 101 can include one or more processors and one or more communication means (e.g., modem) for receiving and transmitting information from and to other systems such as those described herein.

It should be understood that the healthcare data stored in the database 101m can be any information related to the healthcare delivery entity, its patients, their conditions and medical history, their billing information, and other such data known to those of skill in the art. In some embodiments, the stored healthcare can be historical claim feed data. Historical claim feed data refers to data that is derived from medical claims submitted by the healthcare delivery entity and/or in connection with patients of the healthcare delivery entity. Medical claims, which can be used to generate or arrive at the historical claim feed data, include information about a patient's visit or interaction with a healthcare delivery entity. Typically, these medical claims are generated for billing purposes—e.g., for the healthcare delivery entity to request payment for service either from a health insurance provider or the patient. Non-limiting examples of information in each claim includes patient details (e.g., name, address, date of birth, place of birth, gender, ethnicity), basic medical data at the time of the relevant visit (e.g., weight, height, blood pressure), reasons for visit (e.g., symptoms, length of symptoms, exposures, degrees of symptoms), services provided (e.g., medications, treatments), diagnoses, prescriptions, and the like.

It should be understood that a healthcare analytics prediction system 101 can be associated with one or more health provider entities. For example, as shown in FIG. 1, the healthcare analytics prediction system 101 is associated with a hospital 102-1 and a surgical center 102-2 (collectively “102”). The healthcare entities 102 can, in some embodiments, be part of or associated with an ACO. In such a configuration, the health provider entities 102 can collect and share data, and the healthcare analytics prediction system can store and/or analyze data for each of the health provider entities 102. The analytics can be provided based on a combination of data from or related to both of the providers 102, or it can be provided individually based on each entity's respective data.

As also shown in FIG. 1, the healthcare analytics prediction system 101 is communicatively coupled, via a network 105-1, to one or more third party systems 103-1 and 103-2 (collectively “103”). Some non-limiting examples of networks that can be used for communications between the end-user systems 120 and the quality measurement system 101 include a local area network (LAN), personal area network (PAN), wide area network (WAN), and the like. The third party systems 103 can be data warehouse, insurance provider systems, systems of claims management entities, or other similar systems or entities known to those of skill in the art that store, generate or provide healthcare data such as claims data. One example of a third party system 103 is a system managed or controlled by the Centers for Medicare and Medicaid Services (CMS). The CMS systems can continuously collects and stores data related to the claims billing for Medicare and Medicaid participants. The CMS systems, or any third-party systems, can transmit to the healthcare analytics prediction system 101 claims data related to its corresponding health provider entities 102.

Further, the healthcare analytics prediction system 101 is communicatively coupled to end-user systems 104-1 and 104-2 (collectively “104”) via a network 105-2. As discussed above, the network 105-2 can be one of a variety of networks known to those of skill in the art. The end-user systems 104 are computing devices operated by end-users to monitor patient conditions and/or obtain predictions of adverse events. Some non-limiting examples of end-user systems 104 include personal computers, laptops, mobile devices, tablets and the like. Although not illustrated in FIG. 1, the end-user systems 104 can have or be associated with input/output devices, including monitors, projectors, speakers, microphones, keyboards, and the like.

In some example embodiments, the users of the end-user systems 104 include C-level members (e.g., chief executive officer (CEO), chief marketing officer (CMO)), executives, and other care management staff of healthcare provider entities (also referred to as healthcare delivery entities or organizations). The users of the end-user systems can monitor patient conditions and predict adverse events, for example, to provide better staffing and resource management. For instance, a hospital's CEO can use the healthcare analytics prediction system 101 to obtain a prediction of patients that will require a procedure necessitating a particular medicine. The CEO can therefore order enough of that medicine to meet the predicted demands. Other examples of end-users corresponding to the end-user systems 104 include doctors, staff and patients (e.g., for entering or submitting healthcare related information) and system administrators (e.g., for maintaining the systems and its model).

### Process

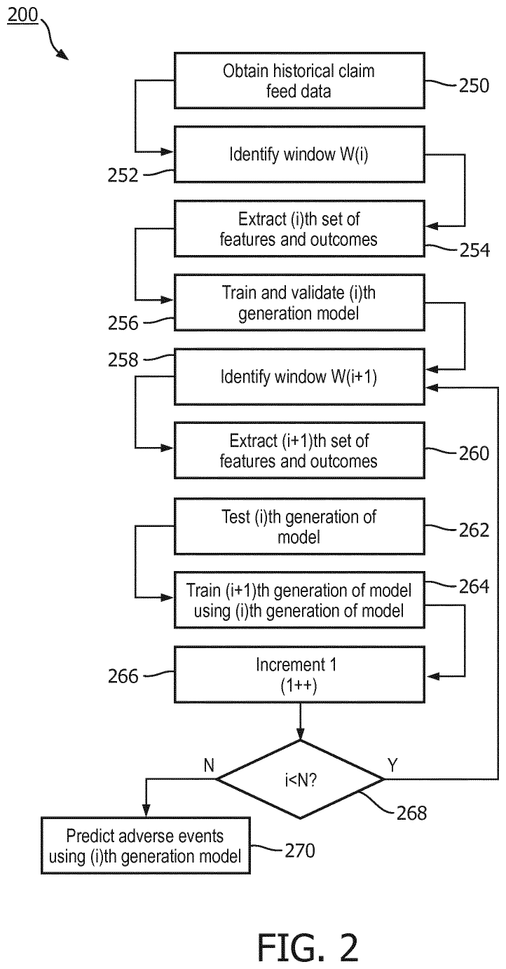

FIG. 2 illustrates a flowchart 200 for dynamically monitoring patient conditions and predicting adverse events. As described above, a healthcare analytics system 101 can dynamically monitor healthcare data and predict adverse events. As described above, the healthcare analytics system 101 can be a system maintained and executed by one or more healthcare provider entities, such as entities associated with an ACO. The system 101 can include or be communicatively coupled to one or more memories or databases that store various healthcare data, including historical claim feed data. The memories or databases (e.g., database 101m) may belong or be managed by the system 101, or can be a separate a third party system (e.g., 103) such as a data warehouse system that stores claim feed data and can, in turn, transmit the claim feed data to the healthcare provider entity. In some embodiments, a database that stores and provides historical claim feed data is maintained by the Centers for Medicare and Medicaid Services (CMS).

As shown in FIG. 2, at step 250, historical claim feed data is received or retrieved by the healthcare analytics system 101. As described above, the historical claim feed data can be obtained from a storage maintained by the healthcare analytics system 101, or from a third party storage such as the CMS's databases. Historical claim feed data is information relating to or derived from patients' healthcare related events and visits to the healthcare provider entity. More specifically, historical claim feed data is made up of a large number of claims associated with the healthcare provider entity corresponding to the healthcare analytics system 101, or the healthcare provider entity's patients.

The claims that make up the historical claim feed data can be generated and/or submitted by the healthcare provider entity, for example, to payer entities such as health insurance providers when seeking payment for healthcare services provided by the healthcare provider entity and detailed in the claims. Each claim in the historical claim feed data can correspond to a patient's visit to the healthcare provider entity, and includes information regarding that visit and data derived therefrom. In some embodiments, the information in a claim includes data regarding the patient's demographics, the healthcare provider entity, and the patient's healthcare.

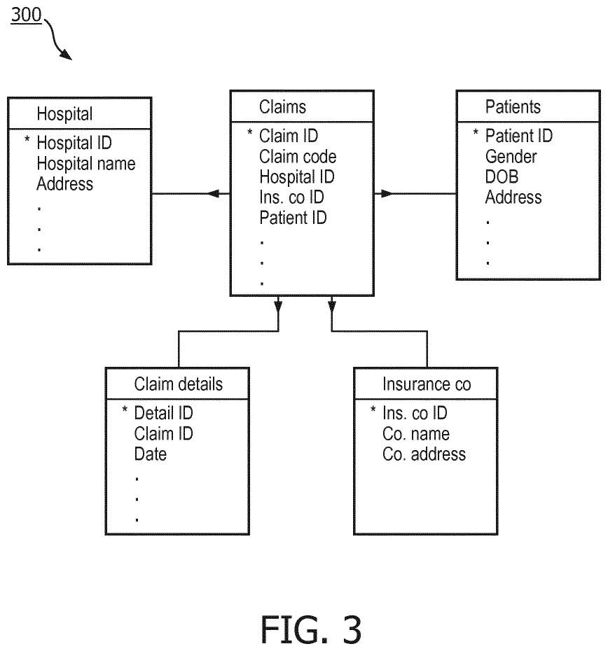

As understood by those of skill in the art, the historical claim feed data that is received or retrieved at step 250 can be in an unstructured or a structured format. Nonetheless, the healthcare analytics system 101 can store the received claim feed data in a structured format, such as in a relational database. FIG. 3 illustrates an example of a data model, including its tables, data and relationships, of a relational database for storing historical claim feed data. As shown in FIG. 3, the historical claim feed data can include information relating to the patient, claims, hospital, staff, insurance policy, prescriptions, services or treatments provided, diagnoses, and others known to those of skill in the art. It should be understood that the historical claim feed data, or any other healthcare data stored by the healthcare analytics system 101, can be stored using any data model known to those of skill in the art.

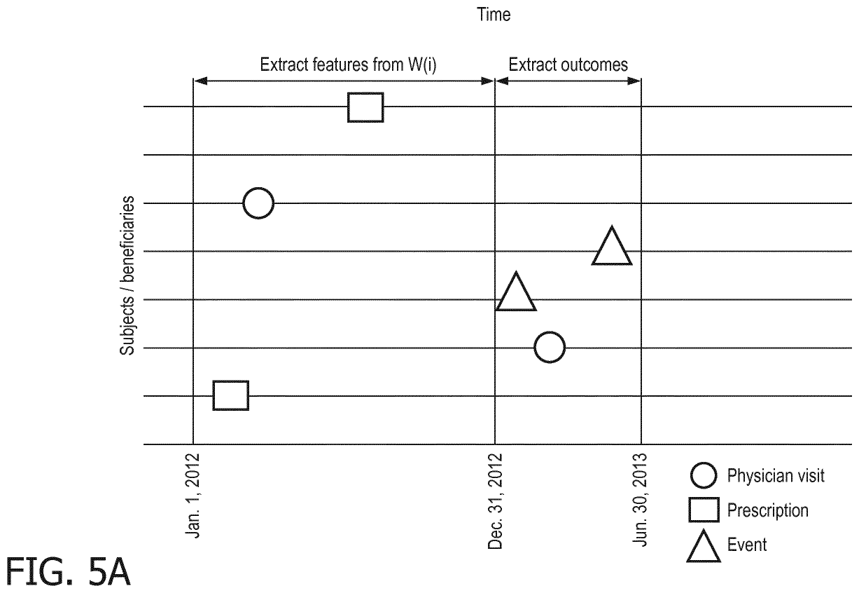

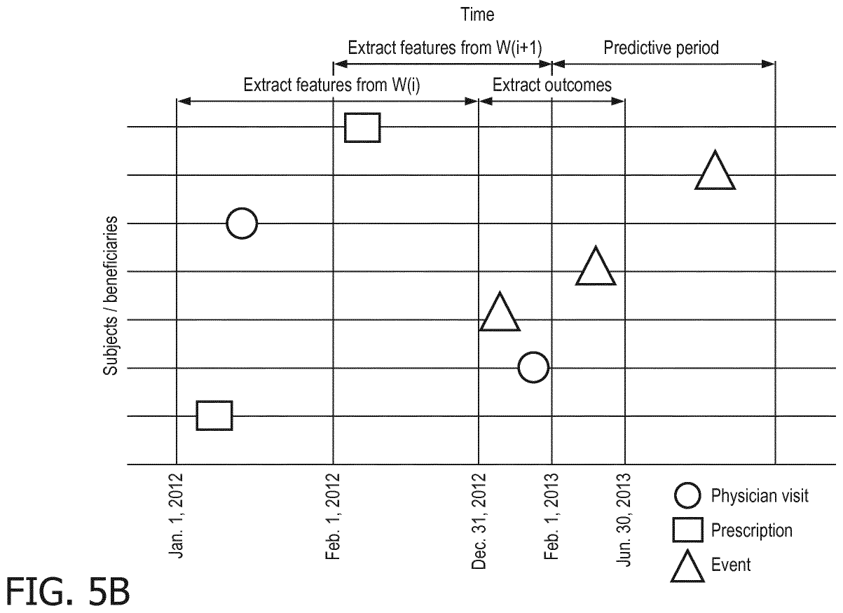

The historical claim feed data received at step 250 is related to claims for a past period of time. FIGS. 5A and 5B, for example, illustrate graphical representations of the historical claim feed data received at step 250 as temporal data. That is, the temporal data representation illustrates the historical claim feed data for each patient (e.g., subject, beneficiary) as a horizontal line. The length of the horizontal lines represents the period of time that the historical claim feed data corresponds to. In one example embodiment, the patient's historical claim feed data spans a past four and a half year period, dating from January 1, 2012 to June 30, 2016. It should be understood, however, that the length of the time period covered by the historical claim feed data can be as short or as long as desired or possible to obtain, though as known to those of skill in the art, historical claim feed data covering a longer period of time and/or more recent time can yield more accurate and/or timely predictions of adverse events.

Still with reference to step 250, the historical claim feed data can be dynamically stored and monitored by the healthcare analytics system 101—e.g., as it is generated. In embodiments in which historical health care data is received by the healthcare analytics system 101, data received or retrieved by the periodically or in a continuous stream (e.g., as the data is generated). For example, in some embodiments in which a third-party system such as the CMS outputs or publishes data periodically (e.g., weekly, monthly), the healthcare analytics system 101 can be configured to receive or retrieve the historical claim feed data each time that it is released by the third-party system. As explained in further detail below, the historical claim feed data received or retrieved at step 250 is used to extract features and outcomes therefrom, and use the extracted features and outcomes to generate models that are used to predict events (e.g., adverse events).

At step 252, an (i)th data chunk referred to as a “window” is identified and prepared for analysis by the healthcare analytics system using a sliding window-based algorithm or approach. This window is also referred to as a current window from among a set of n windows that make up the historical claim feed data. It should be understood that a window refers to a subset of the historical claim feed data that corresponds to a sub-period of time among the period of time covered by the historical claim feed data. The length of the sub-period of time can be any period of time (e.g., one month, six months, one year) deemed optimal or selected by the healthcare analytics system 101.

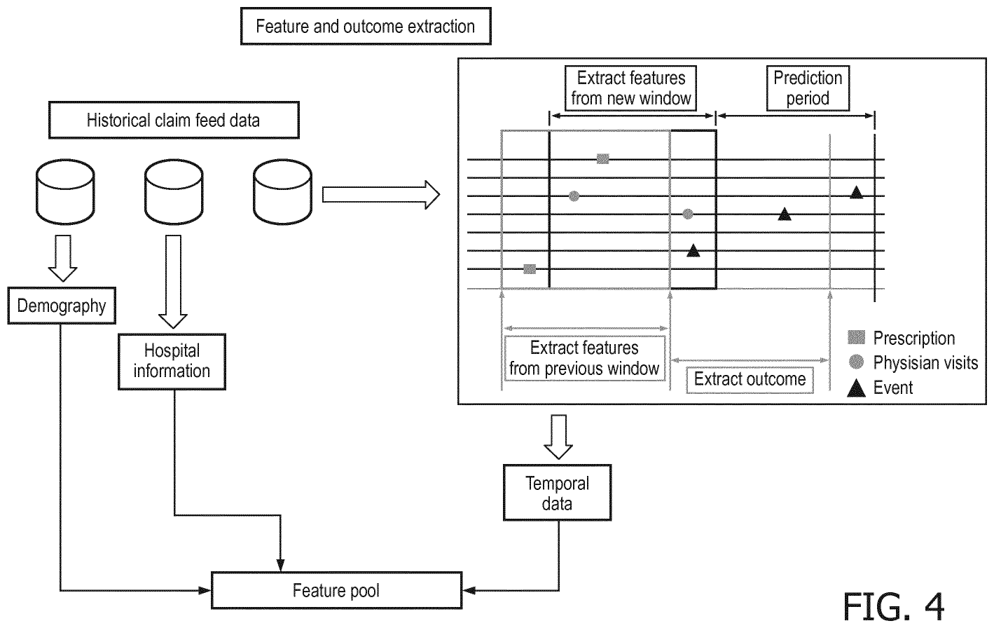

For instance, as shown in exemplary FIG. 4, the historical claim feed data covers the four and a half year period of Jan. 1, 2012 to Jun. 30, 2016. In an exemplary embodiment in which the selected length of each sub-period covered by a window in the sliding window approach is one year, the first window (i=1) in a first iteration covers or corresponds to the sub-period of Jan. 1, 2012 to Dec. 31, 2012. FIG. 4 illustrates, among other windows, the (i)th window W(i), which in an exemplary first iteration in which i=1 is the W(i=1)th window which covers the Jan. 1, 2012 to Dec. 31, 2012 sub-period and its historical claim feed data.

In turn, once the window W(i) has been identified at step 252, an (i)th set of features and outcomes are extracted at step 254. FIG. 5A graphically illustrates the extraction of the (i)th set of features and outcomes from the historical claim feed data. It should be understood that the extracted features can be any data from among the stored or received healthcare data, as selected by the healthcare provider entity or entities associated with the healthcare analytics system 101. In other words, each healthcare analytics system 101 can be configured to extract certain features and not others. This can be based on prior knowledge of features that can have an impact versus features previously deemed impactful on an outcome. For instance, the extracted features can include patient demographic information (e.g., age, gender, weight, height, ethnicity, residence, distance from hospital, etc.) and hospital information (e.g., location, doctors, staff, machinery) during the time period of window W(i) (e.g., Jan. 1, 2012 to Dec. 31, 2012, in an embodiment in which i=1).

Outcomes are also extracted at step 254. The extracted outcomes can include the occurrence of events (e.g., remission, readmission, etc.), healthcare delivery entity visits (e.g., hospital visits, physician visits), or prescriptions provided. However, it should be understood that the outcomes that are extracted can be configured for each system 101 as deemed appropriate, optimal, or necessary. In some embodiments, outcomes are extracted for a period of time of a predetermined length subsequent to the current, (i)th window W(i). For instance, if the desired or optimal period of time for which to extract outcomes is determined to be six months, then, at step 254, the historical claim feed data is analyzed to identify outcomes that occurred in the six month period following W(i). In an exemplary first iteration in which i=1, the sixth month period following the window W(i=1) from which outcomes are extracted is Jan. 1, 2013 to Jun. 30, 2013. The extracted (i)th set of outcomes are graphically represented in the temporal data representation of FIG. 5A for the current, (i)th window.

FIG. 4 is a graphical representation of the extraction of features and outcomes as described above in connection with step 254. As shown in FIG. 4, features can be extracted from the historical claim feed data. In some embodiments, the features can be separated into demographic data, hospital information, and temporal data (e.g., outcomes). These features (and outcomes) can be compiled into a feature (and candidate) pool that includes potential features and outcomes for rapid future identification.

In turn, at step 256, an (i)th generation model is trained using the extracted features and outcomes of step 254. It should be understood that various machine learning or predictive analysis algorithms can be used to train the (i)th generation model, including a Bayesian survival analysis algorithm, online survival LASSO algorithm, and online random survival forest algorithms, as well as other predictive analysis algorithms known to those of skill in the art.

Although training the model can be performed in many ways known to those of skill in the art, in some example embodiments, to train the (i=1)th generation model, the importance of features is determined and/or weights are assigned to one or more of the identified features based on their apparent impact on outcomes within that particular (i)th window W(i). That is, for each of the outcomes of the (i)th set of extracted outcomes, the system 101 analyzes the features of the (i)th set of extracted features to identify patterns. These patterns may be, for example, patterns showing that certain features (or certain values for certain types of features) are commonly associated with a given outcome. For instance, the system 101 can analyze the features and determine that a large number of patients residing in a particular neighborhood suffered respiratory issues. This is interpreted by the system as the outcome of respiratory-related visits, or the like, being largely impacted by the feature of a patient's residence or address. Moreover, for instance, if an outcome is a hospital admission for depression, then all instances of that outcome in the (i)th set of extracted features and outcomes are analyzed to determine which features are most common. For example, if 90% of the instances of hospital admission for depression occur to males between the ages of 50 and 60, then the demographic features of age and gender are deemed to be of higher importance for prediction. Thus, for each specific window and corresponding model, features that are associated with an outcome and that are determined to have an impact on an outcome are deemed to be important variables and treated as predictive variables. For each predictive variable corresponding to the (i)th window W(i), a respective weight is calculated based on the extracted data, and the weight is assigned,based on the predictive variables' calculated impact on an outcome within the the (i)th window W(i). Predictive values from the (i)the window that are given a higher weight in the (i)th generation model are those that frequently appear in connection with a particular outcome in the (i)th window, whereas those features or predictive values that are not frequently associated with the outcome are given a lower weight. It should be understood that, in some embodiments, the importance or weight of variables in one window does not necessarily impact or change the importance or weight of those same variables in other windows.

Still with reference to step 256, once the (i)th generation model has been trained, it can be validated for the six month period following the (i)th window W(i). Validating the (i)th generation model can be performed by running the model against the data of the window W(i) and the features extracted therefrom, and observing whether and/or to what extent the predicted outcomes for the six month period following the window W(i) match the outcomes that actually occurred and that are recorded in the historical claim feed data.

In turn, at step 258, a window W(i+1) is identified or retrieved from among the historical claim feed data. FIG. 5B graphically illustrates the window W(i+1) being identified among the historical claim feed data. The identification of the window at step 258 is similar to that of step 252, in which the window W(i) is identified. As described above, windows refer to sub-periods of time separated by a fixed interval of time. Accordingly, for windows of one year that are separated by one month increments, the window W(i+1) corresponds to the time period beginning and ending one month after the start of the window W(i). Thus, in an initial exemplary implementation in which i=1 as described above, the window W(i+1) refers to a sub-period of time from Feb. 1, 2012 to Jan. 31, 2013.

Similar to step 256, at step 260, an (i+1)th set of features and outcomes is extracted from or in relation to the window W(i+1). FIG. 5B graphically illustrates the extracted (i+1)th set of features and outcomes That is, the extracted features correspond to the period of window W(i+1), and the extracted outcomes correspond to the six month period following the window W(i+1). Thus, in an exemplary first iteration in which i=1, the extracted features for the window W(i+1) correspond to the sub-period of time from Feb. 1, 2012 to Jan. 31, 2013, and the extracted outcomes correspond to the subsequent six month sub-period of time from Feb. 1, 2013 to Aug. 1, 2013.

At step 262, the (i)th generation model is tested against the data of the window W(i+1), to determine the accuracy of the (i)th generation model. More specifically, the (i)th generation model is run against the data and extracted features of the window W(i+1). The outcomes predicted by running the (i)th generation model against the window W(i+1) are compared to the actual outcomes of the sixth month period following the window W(i+1)—e.g., the extracted outcomes from the (i+1)th set of extracted features and outcomes. A testing error rate is identified based on this comparison. The testing error rate is a value indicating the differences or similarities between predicted and actual outcomes In other words, if the predicted outcomes are the same as the outcomes that actually occurred, it can be said that the testing error rate is 0%. The testing error rate can be calculated for every (i)th generation model to ensure that each successive generation of the model improves. In other words, as the model advances and new generations thereof are trained, the testing error rate should continue to increase.

In turn, at step 264, an (i+1)th generation model is generated and/or trained. In some embodiments, the (i+1)th generation model is trained based on or by updating the (i)th generation model using the (i+1)th set of features and outcomes extracted at step 260. As described above, training the (i+1)th generation model can be performed using various techniques and algorithms known to those of skill in the art. In some embodiments, training the (i+1)th generation model is performed by modifying weights and relationships of features relative to those calculated in connection with the (i)th generation model. For instance, if it is determined based on the (i+1)th set of extracted features and outcomes that only a total of 60% of the instances of hospital admissions for depression among (i+1)th set of extracted outcomes were associated with males between the ages of 50 and 60 (as compared to 90% in the ((i)th set of extracted outcomes), then the weight of the age and/or gender features can be reduced in the (i+1)th generation of the model. In this way, the system can continue to evolve as additional historical claim feed data is analyzed.

It should be understood that this above-described analysis of windows one after the other is referred to as a “sliding window” approach.

Once the (i+1)th generation of the model has been trained, the system can determine whether other windows within the historical claim feed data remain to be processed. More specifically, at step 266, the healthcare predictive analysis system 101 increments the value of i by 1 (i++) and, at step 268, determines if i<N. In other words, at steps 266 and 268, the system 101 determines whether windows within the historical claim feed data have not yet been used to train a new generation of the model. These steps ensure that the latest full windows of data are used for the latest generation of the model, such that the model can be as accurate and up-to-date as possible when later used to predict outcomes.

Still with reference to step 268, if the healthcare predictive analysis system 101 determine at step 268 that i<N, and thus windows remain to be processed within the set of N windows, the subsequent window W(i+1) is identified at step 258. It should be understood that, because the value of i was incremented at step 266, the new window W(i+1) refers to the window subsequent to the last window used to train the model. Steps 260, 262 and 264 are repeated in connection with the new window W(i+1).

The healthcare predictive analysis system 101 engages in the loop between steps 258 and 268 until it determines, at step 268, that i>=N, indicating that the last one year length window of data has been processed. Accordingly, in turn, at step 270, the latest generation of the model is used to predict adverse events. That is, at step 270, the healthcare analytics system uses the latest and most up-to-date generation of the model−i.e., the (i)th generation−to determine whether and the probability or likelihood of outcomes occurring at a future time (e.g., instant risk). The (i)the generation of the model is applied to the part of the historical claim feed data does not complete a full window (e.g., a partial window), or to later-acquired data, and the features extracted therefrom.

For instance, at step 270, the (i)th generation of the model is applied to a set of features in partial historical claim feed data, to predict adverse events expected to occur at a later time (e.g., within a six month window following the partial historical claim feed data).

Although not illustrated in FIG. 2, as historical claim feed data is subsequently obtained or received (e.g., from a third party system), the model can be further updated. For example, if at step 270 a partial window of 10 months' worth of data remains to be used to train a new generation of the model, the system can continue to receive historical claim feed data until historical claim feed data is available for the entire one year period, in accordance with the size of each window. At that time, steps 258 to 270 can be repeated using the new window, window W(i+1).

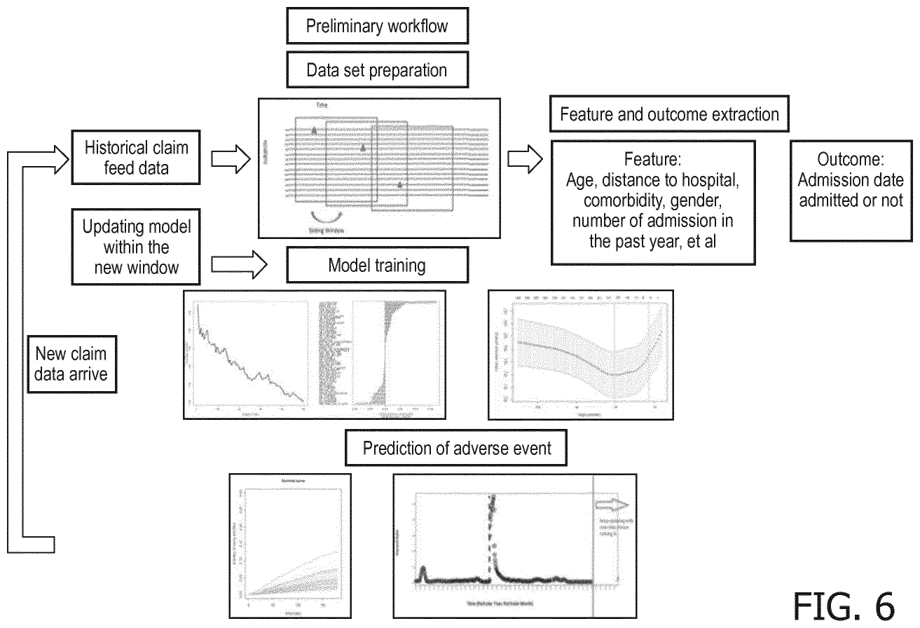

FIG. 6 illustrates a graphical representation of the predictive analysis process described above in connection with FIG. 2. As shown in FIG. 6, historical claim feed data is received, and the data set is prepared by identifying windows or data sets therein of a predetermined length. Features and outcomes are extracted from each of the windows. For each window, a model is trained (or updated) based on the features and outcomes extracted in connection therewith. And, in turn, adverse events are predicted based on the execution of the latest model.

The present embodiments described herein can be implemented using hardware, software, or a combination thereof, and can be implemented in one or more computing device, mobile device or other processing systems. To the extent that manipulations performed by the present invention were referred to in terms of human operation, no such capability of a human operator is necessary in any of the operations described herein which form part of the present invention. Rather, the operations described herein are machine operations. Useful machines for performing the operations of the present invention include computers, laptops, mobile phones, smartphones, personal digital assistants (PDAs) or similar devices.

The example embodiments described above, including the systems and procedures depicted in or discussed in connection with FIGS. 1-7, or any part or function thereof, may be implemented by using hardware, software or a combination of the two. The implementation may be in one or more computers or other processing systems. While manipulations performed by these example embodiments may have been referred to in terms commonly associated with mental operations performed by a human operator, no human operator is needed to perform any of the operations described herein. In other words, the operations may be completely implemented with machine operations. Useful machines for performing the operation of the example embodiments presented herein include general purpose digital computers or similar devices.

Portions of the example embodiments of the invention may be conveniently implemented by using a conventional general purpose computer, a specialized digital computer and/or a microprocessor programmed according to the teachings of the present disclosure, as is apparent to those skilled in the computer art. Appropriate software coding may readily be prepared by skilled programmers based on the teachings of the present disclosure.

Some embodiments may also be implemented by the preparation of application-specific integrated circuits, field programmable gate arrays, or by interconnecting an appropriate network of conventional component circuits.

Some embodiments include a computer program product. The computer program product may be a non-transitory storage medium or media having instructions stored thereon or therein which can be used to control, or cause, a computer to perform any of the procedures of the example embodiments of the invention. The storage medium may include without limitation a floppy disk, a mini disk, an optical disc, a Blu-ray Disc, a DVD, a CD or CD-ROM, a micro-drive, a magneto-optical disk, a ROM, a RAM, an EPROM, an EEPROM, a DRAM, a VRAM, a flash memory, a flash card, a magnetic card, an optical card, nanosystems, a molecular memory integrated circuit, a RAID, remote data storage/archive/warehousing, and/or any other type of device suitable for storing instructions and/or data.

Stored on any one of the non-transitory computer readable medium or media, some implementations include software for controlling both the hardware of the general and/or special computer or microprocessor, and for enabling the computer or microprocessor to interact with a human user or other mechanism utilizing the results of the example embodiments of the invention. Such software may include without limitation device drivers, operating systems, and user applications. Ultimately, such computer readable media further includes software for performing example aspects of the invention, as described above.

Included in the programming and/or software of the general and/or special purpose computer or microprocessor are software modules for implementing the procedures described above.

While various example embodiments of the invention have been described above, it should be understood that they have been presented by way of example, and not limitation. It is apparent to persons skilled in the relevant art(s) that various changes in form and detail can be made therein. Thus, the disclosure should not be limited by any of the above described example embodiments, but should be defined only in accordance with the following claims and their equivalents.

In addition, it should be understood that the figures are presented for example purposes only. The architecture of the example embodiments presented herein is sufficiently flexible and configurable, such that it may be utilized and navigated in ways other than that shown in the accompanying figures.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that the procedures recited in the claims need not be performed in the order presented.

What is claimed is:

1. A healthcare predictive analysis system, comprising:
at least one memory operable to store a set of historical data corresponding to a period of time previous to a present time at a time of execution;
at least one processor communicatively coupled to the at least one memory, the at least one processor being operable to:
retrieve the set of historical data from the at least one memory;
identify a plurality of windows among the set of historical data, each of the plurality of windows being a subset of the set of historical data corresponding to a sub-period of time among the period of time;
identify a current window from among the plurality of windows;
for each of the windows among the plurality of windows:
extract a current set of features and outcomes corresponding to the current window, the current features being extracted from the sub-period of time corresponding to the current window and the current outcomes being extracted from a current outcomes sub-period of time subsequent to the sub-period of time corresponding to the current window;
train a current generation predictive model based on the extracted current set of features and outcomes, the current generation predictive model corresponding to the current window;
identify a next window from among the plurality of windows, the next window being the next-in-time window relative to the current window;
extract a next set of features and outcomes corresponding to the next window, the next features being extracted from the sub-period of time corresponding to the next window and the next outcomes being extracted from a next outcomes sub-period of time subsequent to the sub-period of time corresponding to the next window;
train a next generation predictive model based on the current generation predictive model and the extracted next set of features and outcomes, the next generation predictive model corresponding to the next window; and
substitute the current window with the next window; and
predict a probability of an occurrence of one or more events using a predictive model corresponding to the current window on a subset of data corresponding to a predictive sub-period of time among the period of time.

2. The system of claim 1, wherein the set of historical data is claim feed data corresponding to a healthcare provider entity.

3. The system of claim 2, wherein at least a portion of the set of historical data is received from a third-party database.

4. The system of claim 3,
wherein the portion of the set of historical data received from the third-party database is unstructured data, and
wherein the at least one processor is operable to structure the unstructured data.

5. The system of claim 1, wherein the sub-periods of time corresponding to the plurality of windows are the same length.

6. The system of claim 1, wherein the predictive model used to predict the probability of the occurrence of the one or more events corresponds to a window corresponding to the sub-period of time closest to the present time.

7. The system of claim 1, wherein each of the extracted current outcomes and next outcomes is associated with a time-to-event variable indicating a length of time from the start of the sub-period of time corresponding to the current window and the next window, respectively.

8. The system of claim 1,
wherein the training of the current generation predictive model includes:
for each of the extracted current outcomes:
identifying, among the extracted current features, patterns related to the given extracted current outcome;
identifying one or more current predictive variables based on the identified patterns related to the given extracted current outcome, each of the one or more current predictive variables being one of the extracted current features; and
assigning weights to each of the one or more current predictive variables based on the identified patterns related to the given extracted current outcome; and
wherein the training of the next generation predictive model includes:
for each of the extracted next outcomes:
identifying, among the extracted next features, patterns related to the given extracted next outcome;
identifying one or more next predictive variables based on the identified patterns related to the given extracted next outcome, each of the one or more next predictive variables being one of the extracted next features; and
assigning weights to each of the one or more next predictive variables based on the identified patterns related to the given extracted next outcome,
wherein if the given extracted next outcome matches one of the extracted current outcomes, the assigning of weights includes updating the weights of each of the one or more current predictive variables corresponding to the one of the extracted current outcomes that match the one or more next predictive variables corresponding to the one of the extracted next outcomes.

9. The system of claim 8,
wherein predicting the probability of an occurrence of one or more events using the predictive model includes:
for each of the one or more events:
identifying one or more relevant outcomes in the predictive model;
identifying the predictive variables related to each of the one or more relevant outcomes;
identifying matching features in the subset of data corresponding to the predictive sub-period of time that match features corresponding to the identified predictive variables related to each of the one or more relevant outcomes; and
calculating a probability of the occurrence of each of the one or more events based on the weights of the respective matching features.

10. The system of claim 8, wherein the predicting of the probability of the occurrence of one or more events is performed for a specified future date or date range.

11. The system of claim 1, wherein the at least one processor is further operable to calculate a testing error rate by executing the current generation model against the extracted next features and outcomes.

12. A method for providing healthcare predictive analysis, comprising:
retrieving a set of historical data stored in at least one memory, the set of historical data corresponding to a period of time previous to a present time at a time of execution;
identifying a plurality of windows among the set of historical data, each of the plurality of windows being a subset of the set of historical data corresponding to a sub-period of time among the period of time;
identifying a current window from among the plurality of windows;
for each of the windows among the plurality of windows:
extracting a current set of features and outcomes corresponding to the current window, the current features being extracted from the sub-period of time corresponding to the current window and the current outcomes being extracted from a current outcomes sub-period of time subsequent to the sub-period of time corresponding to the current window;
training a current generation predictive model based on the extracted current set of features and outcomes, the current generation predictive model corresponding to the current window;
identifying a next window from among the plurality of windows, the next window being the next-in-time window relative to the current window;
extracting a next set of features and outcomes corresponding to the next window, the next features being extracted from the sub-period of time corresponding to the next window and the next outcomes being extracted from a next outcomes sub-period of time subsequent to the sub-period of time corresponding to the next window;
training a next generation predictive model based on the current generation predictive model and the extracted next set of features and outcomes, the next generation predictive model corresponding to the next window; and
substituting the current window with the next window; and
predicting a probability of an occurrence of one or more events using a predictive model corresponding to the current window on a subset of data corresponding to a predictive sub-period of time among the period of time.

13. The method of claim 12, wherein the set of historical data is claim feed data corresponding to a healthcare provider entity.

14. The method of claim 13, wherein at least a portion of the set of historical data is received from a third-party database.

15. The method of claim 14,
wherein the portion of the set of historical data received from the third-party database is unstructured data, and
wherein the method further comprises structuring the unstructured data.

16. The method of claim 12, wherein the sub-periods of time corresponding to the plurality of windows are the same length.

17. The method of claim 12, wherein the predictive model used to predict the probability of the occurrence of the one or more events corresponds to a window corresponding to the sub-period of time closest to the present time.

18. The method of claim 12, wherein each of the extracted current outcomes and next outcomes is associated with a time-to-event variable indicating a length of time from the start of the sub-period of time corresponding to the current window and the next window, respectively.

19. The method of claim 12,
wherein the training of the current generation predictive model includes:
for each of the extracted current outcomes:
identifying, among the extracted current features, patterns related to the given extracted current outcome;
identifying one or more current predictive variables based on the identified patterns related to the given extracted current outcome, each of the one or more current predictive variables being one of the extracted current features; and
assigning weights to each of the one or more current predictive variables based on the identified patterns related to the given extracted current outcome; and
wherein the training of the next generation predictive model includes:
for each of the extracted next outcomes:
identifying, among the extracted next features, patterns related to the given extracted next outcome;
identifying one or more next predictive variables based on the identified patterns related to the given extracted next outcome, each of the one or more next predictive variables being one of the extracted next features; and
assigning weights to each of the one or more next predictive variables based on the identified patterns related to the given extracted next outcome,
wherein if the given extracted next outcome matches one of the extracted current outcomes, the assigning of weights includes updating the weights of each of the one or more current predictive variables corresponding to the one of the extracted current outcomes that match the one or more next predictive variables corresponding to the one of the extracted next outcomes.

20. The method of claim 19,
wherein predicting the probability of an occurrence of one or more events using the predictive model includes:
for each of the one or more events:
identifying one or more relevant outcomes in the predictive model;
identifying the predictive variables related to each of the one or more relevant outcomes;
identifying matching features in the subset of data corresponding to the predictive sub-period of time that match features corresponding to the identified predictive variables related to each of the one or more relevant outcomes; and
calculating a probability of the occurrence of each of the one or more events based on the weights of the respective matching features.

21. The method of claim 19, wherein the predicting of the probability of the occurrence of one or more events is performed for a specified future date or date range.

22. The method of claim 12, further comprising calculating a testing error rate by executing the current generation model against the extracted next features and outcomes.
